(19) United States
(12) Patent Application Publication
(10) Pub. No.: US 2010/0035309 A1
(43) Pub. Date: Feb. 11, 2010

(54) ANALYSIS AND ENHANCEMENT OF METABOLIC PATHWAYS FOR METHANOGENESIS

(75) Inventors: Shelley Havemen, Lakewood, CO (US); Gary Vanzin, Arvada, CO (US); Glenn Ulrich, Rolla, MO (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: LUCA Technologies, Inc., Golden, CO (US)

(21) Appl. No.: 12/187,274

(22) Filed: Aug. 6, 2008

(51) Int. Cl. C12P 17/18; C12P 5/00

(52) U.S. Cl. 435/119; 435/166

(57) ABSTRACT

Processes for biogenic production of a hydrogen-carbon-containing fluid from a hydrocarbon containing formation are described. The processes may include providing in the formation an anaerobic microorganism consortium containing one or more enzymes to activate a starting hydrocarbon by an addition of a chemical group to the hydrocarbon. The processes may further include converting the activated hydrocarbon into the hydrogen-carbon-containing fluid through one or more intermediate hydrocarbons, and recovering the hydrogen-carbon-containing fluid from the formation.

### FIELD OF THE INVENTION

The present invention relates to biogenic enhancement of the mole percentage of hydrogen in hydrocarbon molecules and enhancements in biogenic hydrogen and methane production in geologic formations. Specifically, the invention relates to providing or stimulating a microorganism consortium having one or more enzymes capable of transforming carbonaceous materials in the formations into hydrogen-carbon-containing fluids (i.e., liquids or gases) such as methane.

### BACKGROUND

Increasing world energy demand is creating unprecedented challenges for recovering energy resources, and mitigating the environmental impact of using those resources. Some have argued that the worldwide production rates for oil and domestic natural gas will peak within a decade or less. Once this peak is reached, primary recovery of oil and domestic natural gas will start to decline, as the most easily recoverable energy stocks start to dry up. Historically, old oil fields and coal mines are abandoned once the easily recoverable materials are extracted. These abandoned reservoirs, however, still contain significant amounts of carbonaceous material. The Powder River Basin in northeastern Wyoming, for example, is still estimated to contain approximately 1,300 billion short tons of coal. Just 1% of the Basin's remaining coal converted to natural gas could supply the current annual natural gas needs of the United States (i.e., about 23 trillion cubic feet) for the next four years. Several more abandoned coal and oil reservoirs of this magnitude are present in the United States.

As worldwide energy prices continue to rise, it may become economically viable to extract additional oil and coal from these formations with conventional drilling and mining techniques. However, a point will be reached where more energy must be used to recover the resources than is gained by the recovery. At that point, traditional recovery mechanisms will become uneconomical, regardless of the price of energy. Thus, new recovery techniques are needed that can extract resources from these formations with significantly lower expenditures of energy.

Conventional recovery techniques also extract the carbonaceous materials in their native state (e.g., crude oil, coal), and the combustion products of these materials may include a number of pollutants, including sulfur compounds (SOₓ), nitrogen compounds (NOₓ) and carbon dioxide (CO₂). Concern about the environmental impact of burning these native carbonaceous materials has led to national and international initiatives to develop less polluting energy sources. One approach is to generate more energy with natural gas (i.e., methane), which has low levels of sulfur and nitrogen, and generates less carbon dioxide per unit energy than larger hydrocarbons.

One alternative to conventional recovery techniques has been to provide or stimulate microorganism in a formation to metabolize the carbonaceous materials into compounds more easily recoverable, such as gaseous methane. These biogenic approaches often involve attempts to identify genera and species of the microorganisms present in a formation that appears to be biogenically active. However, the microorganisms involved in biogenic production have not been exhaustively catalogued. Furthermore functionality is not always linked to the identity of the microorganisms. Thus, an analysis of biogenic production processes that tries to identify all genera and species of microorganisms present in a formation is often incomplete. Accordingly, additional methods of characterizing the biogenic activity of a microorganism consortium may be desired for a more complete and accurate understanding of biogenic production processes in a hydrocarbon containing formation.

### BRIEF SUMMARY

Biological analyses of the microorganisms involved in the biogenic conversion of formation hydrocarbons to simpler hydrogen-carbon-containing fluids (where fluids can be liquids, gases, or both) like methane have primarily focused on identifying the genera and species of microorganisms found at the center of the activity. Processes described here include analyses of the enzymes used by the microorganisms to catalyze these biogenic conversions. The genes encoding these enzymes, as well as the enzymes themselves, may be found in microorganisms of different genera and/or species. Thus, the identification of the genes and enzymes can suggest a broader range of microorganism genera and species that may be assembled into a productive consortium for the biogenic conversion (i.e., degradation) of the formation hydrocarbons. It may also suggest microorganism genera and/or species that are more compatible with the consortium to provide a key enzyme (or set of enzymes) in the biogenic conversion. Thus, by identifying the enzymes (and the genes that encode them) involved in the biogenic conversion pathways more reliable information can be obtained on enhancing the biogenic production of hydrogen-carbon-containing fluids.

This information may include collecting proteomic and genomic data on the types and relative concentrations of the enzymes present in a microorganism consortium in a formation. This enzymatic data may then be analyzed against known enzymatic pathways to convert formation hydrocarbons like coal and oil into hydrogen-carbon-containing fluids like methane. The consortium enzyme analyses may identify bottlenecks in enzymatic pathways that may not be apparent from a genus/species identification. Knowledge of these enzymatic bottlenecks may be used to design changes to the consortium makeup and/or environmental conditions in the formation to partially or completely remove them.

Embodiments include processes for biogenic production of a hydrogen-carbon-containing fluid from a hydrocarbon containing formation. The processes may include providing in the formation an anaerobic microorganism consortium containing one or more enzymes to activate a starting aromatic hydrocarbon by an addition of a chemical group to the starting aromatic hydrocarbon. The processes may further include converting the activated aromatic hydrocarbon into the hydrogen-carbon-containing fluid through one or more intermediate hydrocarbons, and recovering the hydrogen-carbon-containing fluid from the formation.

Embodiments further include additional processes for biogenic production of a hydrogen-carbon-containing fluid from a hydrocarbon containing formation. The processes may include stimulating in the formation an anaerobic microorganism consortium containing one or more enzymes to activate a starting aromatic hydrocarbon by an addition of a chemical group to the starting aromatic hydrocarbon. The processes may further include converting the activated aromatic hydrocarbon into the hydrogen-carbon-containing fluid through one or more intermediate hydrocarbons, and recovering the hydrogen-carbon-containing fluid from the formation.

Embodiments may still further include additional processes for biogenic production of a hydrogen-carbon-containing fluid from a hydrocarbon containing formation. The processes may include providing to the formation an anaerobic microorganism consortium containing one or more enzymes to activate a starting alkane into an activated hydrocarbon by an addition of a chemical group to the starting alkane. The processes may also include converting the activated hydrocarbon into the hydrogen-carbon-containing fluid through one or more intermediate hydrocarbons, and recovering the hydrogen-carbon-containing fluid from the formation.

Embodiments may also further include still additional processes for biogenic production of a hydrogen-carbon-containing fluid from a hydrocarbon containing formation. The processes may include stimulating in the formation an anaerobic microorganism consortium containing one or more enzymes to activate a starting alkane into an activated hydrocarbon by an addition of a chemical group to the starting alkane. The processes may also include converting the activated hydrocarbon into the hydrogen-carbon-containing fluid through one or more intermediate hydrocarbons, and recovering the hydrogen-carbon-containing fluid from the formation.

Additional embodiments and features are set forth in part in the description that follows, and in part will become apparent to those skilled in the art upon examination of the specification or may be learned by the practice of the invention. The features and advantages of the invention may be realized and attained by means of the instrumentalities, combinations, and methods described in the specification.

### BRIEF DESCRIPTION OF THE DRAWINGS

A further understanding of the nature and advantages of the present invention may be realized by reference to the remaining portions of the specification and the drawings wherein like reference numerals are used throughout the several drawings to refer to similar components. In some instances, a sublabel is associated with a reference numeral and follows a hyphen to denote one of multiple similar components. When reference is made to a reference numeral without specification to an existing sublabel, it is intended to refer to all such multiple similar components.

FIG. 1 shows selected steps in a process for the biogenic production of a hydrogen-carbon-containing fluid according to embodiments of the invention;

FIG. 2 shows selected steps in another process for the biogenic production of hydrogen-carbon-containing fluids according to embodiments of the invention;

FIG. 3 shows selected steps in an enzyme analysis and enzymatic pathway optimization process according to embodiments of the invention;

FIGS. 4A & B show an exemplary enzymatic pathway for the anaerobic metabolism of a starting aromatic hydrocarbon to methane; and

FIGS. 5A & B show an exemplary enzymatic pathway for the anaerobic metabolism of a starting non-cyclic hydrocarbon to methane.

### DETAILED DESCRIPTION

Enzymes and processes are described for producing hydrogen-carbon-containing fluids (e.g., methane) from a hydrocarbon formation (e.g., an oil field, coal bed, etc.) with the help of an anaerobic microorganism consortium that has one or more enzymes to catalyze the transformation of starting hydrocarbons in the formation into the hydrogen-carbon-containing fluids. The processes may include introducing a consortium with these enzymes into the formation to initiate or enhance biological catalytic activity that produces the hydrogen-carbon-containing fluids.

In many instances, microorganisms in the consortium use the hydrocarbons in the formation as growth substrates, and transform the starting hydrocarbons through one or more metabolic pathways into the hydrogen-carbon-containing fluid and energy. Present processes may include identifying enzymes involved in the metabolic pathway and providing a microorganism consortium that includes one or more microorganisms that individually or collectively have all the enzymes needed to complete the pathway. Some of these enzymes may be identified in microorganisms already present in the formation, while others may be provided to the formation.

While the final hydrogen-carbon-containing fluids may represent an endpoint for a metabolic pathway, they may still represent useful fuels for heating, transportation, and electricity generation, among other uses. When a metabolic pathway transforms difficult to recover solid and tar-like hydrocarbons into more easily recoverable gases and liquids (i.e. fluids), the processes described here can extend the productive lifetime of an oil, gas, or coal formation for several additional years.

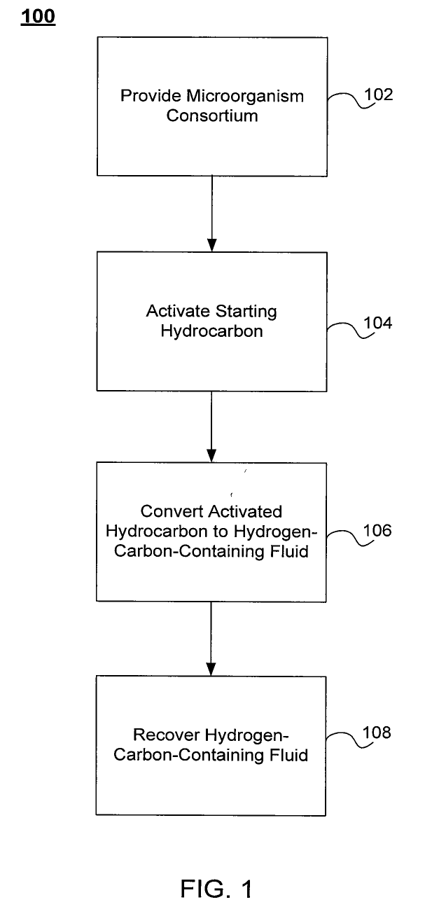

Referring now to FIG. 1 selected steps in a process for the biogenic production of a hydrogen-carbon-containing fluid according to embodiments of the invention is shown. The process 100 may include providing an microorganism consortium 102 in a hydrocarbon containing formation. The consortium may be provided by stimulating in situ growth of selected microorganisms in the formation, and/or transporting microorganism into the formation from an external source (which may include one or more other hydrocarbon-containing formations). The consortium includes one or more enzymes that activate a starting hydrocarbon 104, for example by the addition of a chemical group. The activated hydrocarbon is then converted to the hydrogen-carbon-containing fluid 106 through one or more intermediate hydrocarbons. The conversion of the activated hydrocarbon through the intermediate hydrocarbons may be catalyzed by additional enzymes present in the microorganism consortium.

The final hydrogen-carbon containing fluid may include liquid or gaseous hydrocarbons that have a greater mol. % of hydrogen atoms than the starting hydrocarbons. The final hydrogen-carbon containing fluid may have fewer C—C bonds and/or more C—H bonds than the starting hydrocarbons, resulting in a higher mol. % of hydrogen atoms due to the increase in the ratio of hydrogen atoms to non-hydrogen atoms (e.g., carbon atoms) in the final fluid. For example, acetic acid has the chemical formula CH₃COOH, representing 2 carbon atoms, 2 oxygen atoms, and 4 hydrogen atoms, to give a total of 8 atoms. Since 4 of the 8 atoms are hydrogen, the mol. % of hydrogen atoms in acetic acid is: (4 Hydrogen Atoms)/(8 Total Atoms)=0.5, or 50%, by mol. (or on a molar basis). Methane has the chemical formula CH₄, representing 1 carbon atom and 4 hydrogen atoms, making a total of 5 atoms. The mol. % of hydrogen atoms in methane is (4 Hydrogen Atoms)/(5 Total Atoms)=0.8, or 80%, by mol. Thus, the conversion of acetic acid to methane increases the mol. % of hydrogen atoms from 50% to 80%. In the case of molecular hydrogen, the mol. % of hydrogen atoms is 100%. The biogenic conversion processes can increase in the mol. % of hydrogen atoms from the starting hydrocarbons to the final hydrogen-carbon containing fluid from, for example, less than about 66% to about 80% or more.

The final hydrogen-carbon containing fluid may also include unsaturated hydrocarbons having smaller numbers of carbon atoms and/or more carbon-carbon double and triple bonds than the starting formation hydrocarbons. For example, the biogenic conversion may take an alkane and convert it into an unsaturated alkene or alkyne having the same or few number of carbon atoms. These final hydrogen-carbon containing fluids may include ethylene and acetylene, among other alkenes and alkynes. The unsaturated final products may also include hydrocarbons with a plurality of double and/or triple carbon-carbon bonds.

The final hydrogen-carbon containing fluid may also include oxygen containing compounds such as, alcohols, alkoxides, ketones, ethers, esters, organic acids, and organic acid anhydrides, among other hydrogen-carbon-oxygen containing compounds.

After the starting hydrocarbon in the formation is enzymatically converted into the final hydrogen-carbon-containing fluid, the fluid may be recovered 108 from the formation. For example, when the hydrogen-carbon-containing fluid is gaseous methane, the methane may be recovered using conventional gas well recovery and transport techniques.

The starting hydrocarbons present in the formation may include coal, oil, kerogen, peat, lignite, oil shale, tar sands, bitumen, and/or tars, among other kinds of hydrocarbons. The geological formation may be classified as an oil formation, a natural gas formation, a coal formation, a bitumen formation, a tar sands formation, a lignite formation, a peat formation, a carbonaceous shale formation, or a formation rich in organic matter, among other types of formations.

When the geologic formation is subterranean, the ambient oxygen concentration is typically below that found in tropospheric air (i.e., about 18%-21%, by mol., free O₂), and often below the minimum concentration for obligate aerobes (i. e., microorganisms that require molecular oxygen) to survive. Thus, the enzymatic pathways described are favored under anaerobic conditions where there is little (if any) competition from aerobic metabolic pathways. The microorganisms that use these enzymatic pathways may be living in formation environments where the O₂ concentration is less than about 10%, by mol., less than about 5%, by mol., less than about 2%, by mol., less than about 0.5%, by mol., etc. When microorganisms are living in formation water, this water may also contain less dissolved oxygen than typically measured for surface water (e.g., about 16 mg/L of dissolved oxygen). For example, the formation water may contain about 1 mg/L or less of dissolved oxygen.

The microorganisms in the consortium may include obligate and/or facultative anaerobes. Obligate anaerobes are anaerobes that cannot survive in an atmosphere with molecular oxygen concentrations that approach those found in tropospheric air (e.g., 18% to 21%, by mol. in dry air), or are microorganisms for which free oxygen is considered toxic. Facultative anaerobes are anaerobes that can adapt to both aerobic and anaerobic conditions. Facultative anaerobes can grow in the presence or absence of oxygen, but grow better in the presence of oxygen. Consortium members may also include microorganisms that are viable under reduced oxygen conditions, even if they prefer or require some oxygen. These members may include microaerophiles that proliferate under conditions of increased carbon dioxide concentrations of about 10%, by mol., or more (e.g., above 375 ppm of CO₂).

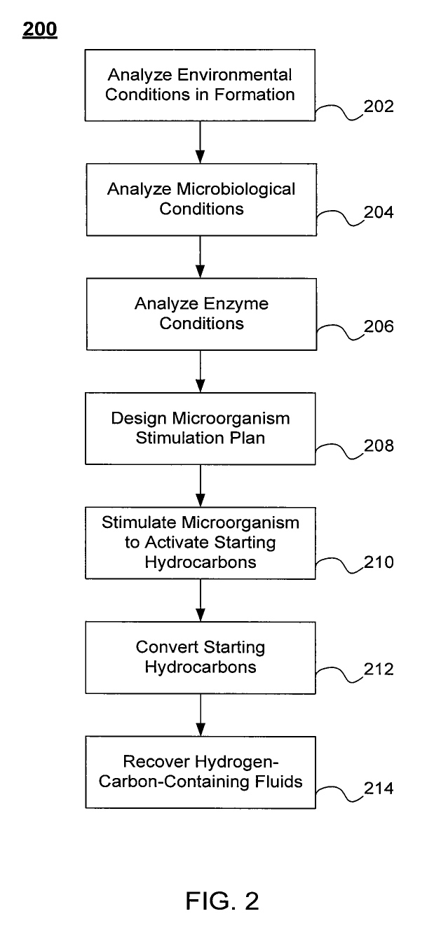

FIG. 2 shows selected steps in another process for the biogenic production of hydrogen-carbon-containing fluids according to embodiments of the invention. The process 200 may include analyzing hydrocarbon compositions including the relative proportion of saturated hydrocarbons (including n-alkanes) and aromatic hydrocarbons, environmental conditions in the formation environment 202, as well as analyzing the microbiological conditions in the formation 204. The environmental conditions analyzed may include temperature, pressure, atmospheric composition, among other conditions. They may also include chemical analyses of the formation such as formation water measurements of pH, salinity, oxidation potential (Eh), an concentrations of elements like dissolved carbon, phosphorous, nitrogen, sulfur, magnesium, manganese, iron, calcium, zinc, tungsten, cobalt and molybdenum (among other elements), as well as concentration measurements for polyatomic ions such as PO₂³⁻, PO₃³⁻, and PO₄³⁻, NH₄⁺, NO₂⁻, NO₃⁻, and SO₄²⁻ (among other ions). The quantities of vitamins, and other microorganism nutrients including element components of cofactors comprising key enzymes involved in anaerobic hydrocarbon biodegradation may also be determined. Additional details of analyses that may be performed are described in co-assigned PCT Application No. PCT/US2005/015259, filed May 3, 2005; and U.S. patent applicaton Ser. No. 11/343,429, filed Jan. 30, 2006, of which the entire contents of both applications are herein incorporated by reference for all purposes.

The biological conditions analyzed may include an identification of the genera and species of microorganisms present in the formation as well as their relative population percentages. These analyses may be performed using an analysis of the DNA of the microorganisms may be done where the DNA is optionally cloned into a vector and suitable host cell to amplify the amount of DNA to facilitate detection. In some embodiments, the detecting is of all or part of ribosomal DNA (rDNA), of one or more microorganisms. Alternatively, all or part of another DNA sequence unique to a microorganism may be detected. Detection may be by use of any appropriate means known to the skilled person. Non-limiting examples include restriction fragment length polymorphism (RFLP) or terminal restriction fragment length polymorphism (TRFLP); polymerase chain reaction (PCR); DNA-DNA hybridization, such as with a probe, Southern analysis, or the use of an array, microchip, bead based array, or the like; denaturing gradient gel electrophoresis (DGGE); or DNA sequencing, including sequencing of cDNA prepared from RNA as non-limiting examples. Quantitative analysis of the relative population percentages may be performed using direct cell counting techniques, including the use of microscopy, flow cytometry, DNA quantification, phospholipid fatty acid analysis, quantitative PCR, protein analysis, etc. Anaerobic metabolism, including methanogenesis and hydrocarbon bioconversion can also be measured. Additional details of the biological analyses are described in co-assigned U.S. patent application Ser. No. 11/099,879, filed Apr. 5, 2005, the entire contents of which is herein incorporated by reference for all purposes.

The process 200 may further include enzyme analyses 206 of microorganism samples collected from a formation environment. The enzyme analyses may include searching for sequences of DNA and/or RNA that encodes for an protein that makes up all or part of an enzyme used in an enzymatic pathway for converting native formation hydrocarbons to a hydrogen-carbon-containing fluid of interest. The enzyme analyses may also include protein analyses that indicate the types and quantities of enzymes that are present in the sample.

After conducting analyses of the environmental, biological, and/or enzymatic conditions present in the formation, a plan may be designed to stimulate 208 in the formation a microorganism consortium having one or more enzymes to activate the conversion of starting hydrocarbons (e.g., coal, oil, tar, aromatic hydrocarbons, non-cyclic hydrocarbons, etc.) into target hydrogen-carbon-containing fluids. The plan may include, changing one or more environmental conditions in the formation, adding a chemical amendment to the formation, adding water to the formation, and/or adding microorganisms to the formation, among other actions.

Implementation of the plan stimulates enzymes in the microorganism consortium to activate a starting hydrocarbon 210. This activation may include the addition to a chemical group (such as a hydrogen, water, a methyl group, an alkyl group, a fumarate group, a carboxyl group, etc.) to the starting hydrocarbon. The activated hydrocarbon may then be converted to the hydrogen-carbon-containing fluid 212 through one or more intermediate hydrocarbons. Enzymes stimulated by the actions of the stimulation plan may catalyze the formation (and/or decomposition) of the intermediate hydrocarbons along the enzymatic pathway to the generation of the final hydrogen-carbon-containing fluid.

After the starting hydrocarbon in the formation is enzymatically converted into the final hydrogen-carbon-containing fluid, the fluid may be recovered 214 from the formation. For example, when the hydrogen-carbon-containing fluid is gaseous methane, the methane may be recovered using conventional gas well recovery and transport techniques.

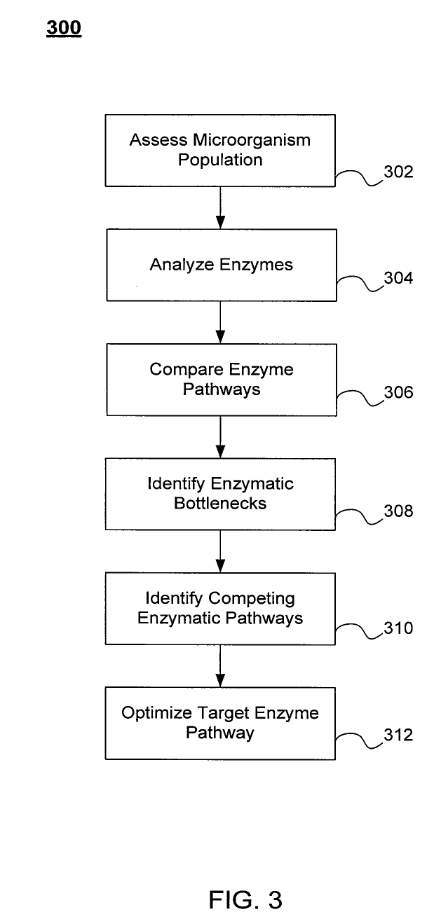

FIG. 3 shows selected steps in an enzyme analysis and enzymatic pathway optimization process according to embodiments of the invention. The process 300 may include assessing a microorganism population 302 in a geologic formation. This assessment may be done in-situ by introducing biological analysis tools into the formation, or samples of the microorganisms may be recovered from the formation and analyzed in a field or off-site laboratory setup. When the microorganisms are predominantly (or exclusively) anaerobic, care should be taken to maintain the anaerobic environment when samples are transported to the surface. Additional details on handling samples of anaerobic microorganisms can be found in co-assigned U.S. patent application Ser. No. 11/399,099 to Pfeiffer et al, filed Apr. 5, 2006 and titled “Chemical Amendments for the Stimulation of Biogenic Gas Generation in Deposits of Carbonaceous Material” the entire contents of which is herein incorporated by reference for all purposes.

The assessment of the formation microorganisms may include an analysis of the identities and activity of the enzymes 304 in the microorganisms that participate in the enzymatic conversion of formation hydrocarbons to hydrogen-carbon-containing fluids of interest. It may also include identifying the types and concentrations of hydrocarbon substrate molecules and metabolic intermediates (e.g., hydrocarbon intermediates) produced by the microorganisms. Accumulation of metabolic intermediates may be an indication of a bottleneck in the metabolic pathway that includes that intermediate. The enzymes and intermediates analyzed may then be compared with known enzyme pathways 306 for these conversions. Missing enzymes, enzymes having low or limiting concentrations, and energetically unfavorable steps in metabolic pathways can be identified. The comparisons may help identify possible bottlenecks 308 in the observed enzymatic pathways of the microorganisms. They may also identify competing enzymatic pathways 310 that metabolize the starting hydrocarbons to different final products.

Based on the analysis, an action plan may be developed to optimize a target enzyme pathway 312. For example, a chemical amendment or an addition of microorganisms may be made to provide or stimulate a microorganism consortium in the formation to favor the desired enzymatic pathway. Alternatively, an amendment may be introduced to discourage the use of a competing enzymatic pathway. By introducing an amendment that will provide energy to the microorganism consortium, thermodynamically unfavorable enzymatic reactions may be catalyzed. Amendments may include trace metal addition based on known metal composition of the targeted enzyme(s). Amendments may also include organic substrate molecules known to support the growth of an identified microorganism(s) detected in the formation that contain the target enzymes. Environmental conditions including pH, salinity, temperature, sulfate, nutrient composition and combinations thereof can be adjusted to target the growth of indigenous formation microorganisms or added microorganisms containing the target enzyme(s).

### Enzymatic Pathways for Conversion of Formation Hydrocarbons

As noted above, the atmosphere in subterranean geologic formations often have a lower concentration of free molecular oxygen (O₂) than tropospheric air. In these anoxic environments, microorganisms may rely on enzymatic pathways for anaerobic metabolism of the hydrocarbons present in the formation. The intermediates and final products of these pathways include hydrogen-carbon-containing fluids, such as C₁₋₆ alkanes, and acetate. They may also include molecular hydrogen (H₂). Described below are some exemplary enzymatic pathways for the anaerobic conversion of starting hydrocarbons into hydrogen-carbon-containing products. These pathways include a plurality of individual enzymes that each play a role in converting the starting hydrocarbon through a series of intermediate hydrocarbons to the final hydrogen-carbon-containing fluid compound.

### Exemplary Enzymatic Pathway for Aromatic Hydrocarbons

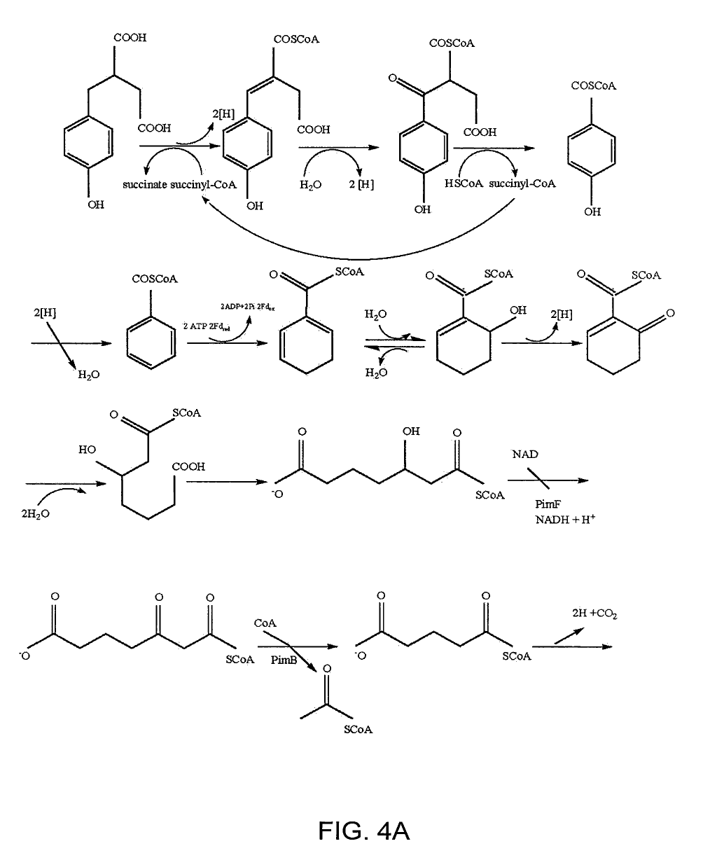

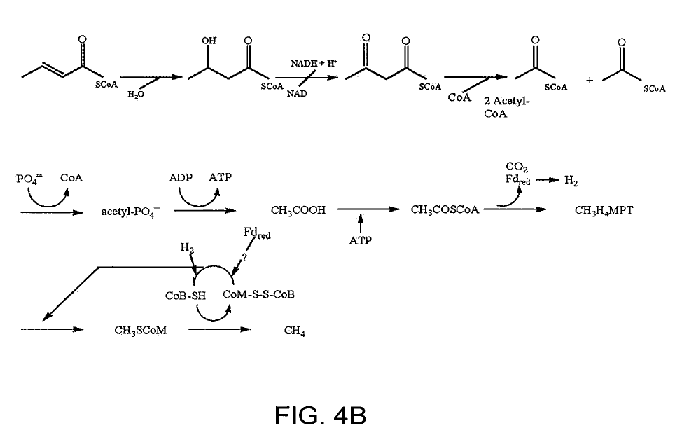

FIG. 4 shows an exemplary enzymatic pathway for the anaerobic metabolism of a starting aromatic hydrocarbon to methane (i.e., the final hydrogen-carbon-containing fluid). The first stages of the pathway include the activation of the starting aromatic hydrocarbon by the addition of a chemical group. For example, fumarate (HO₂CCH═CHCO₂H) may be added to an alkyl group on an aromatic ring (e.g., toluene, ethylbenzene, etc.) to make an activated aromatic hydrocarbon. In the case of toluene as the starting aromatic hydrcarbon, a benzylsuccinate synhtase enzyme catalyzes the fumarate addition to the methyl group.

The next stages in the pathway may include the enzymatic conversion of the activated aromatic hydrocarbon to benzoyl-CoA, a central intermediate of anaerobic aromatic metabolism. One exemplary metabolic pathway enzymatically converts the activated aromatic hydrocarbon (e.g., p-hydroxybenzylsuccinate) through a series of β-oxidation-like intermediates to p-hydroxybenzoyl-CoA. These steps may start with the conversion of the p-hydroxybenzylsuccinate to p-hydroxybenzylsuccinate-CoA using a p-hydroxybenzylsuccinate-CoA transferase enzyme to catalyze the transfer a CoA group from succinyl-CoA, and a p-hydroxybenzylsuccinate-CoA dehydrogenase. The p-hydroxybenzylsuccinate-CoA may then be converted into a p-hydroxyphenylitaconyl-CoA through the enzymatic conversion of a p-hydroxyphenylitaconyl-CoA hydratase enzyme and a 3-hydroxyenoyl-CoA dehydrogenase enzyme. The p-hydroxyphenylitaconyl-CoA may then be convered to p-hydroxybenzoyl-CoA by a benzoylsuccinyl-CoA thiolase enzyme. Finally, the p-hydroxybenzoyl-CoA is converted to benzoyl-CoA with a p-hydroxybenzoyl-CoA reductase enzyme.

The next stages may include an enzymatic pathway that opens the aromatic ring in benzoyl-CoA to form a hydroxypimelyl-CoA compound. This benzoyl-CoA degradation pathway may start with the conversion of benzoyl-CoA into cyclohexa-1,5-diene-1-carbonyl-CoA through enzymatic conversion using a benzoyl-CoA reductase enzyme. The cyclohexa-1,5-diene-1-carbonyl-CoA may then be converted to 6-hydroxycyclohex-1-ene-1-carbonyl-CoA through enzymatic conversion using cyclohexa-1,5-diene-1-carbonyl-CoA hydratase enzyme. The 6-hydroxycyclohex-1-ene-1-carbonyl-CoA may then be converted to 6-oxocyclohex-1-ene-1-carbonyl-CoA (i.e., 6-OCH-CoA) through enzymatic conversion using a 6-hydroxycyclohex-1-ene-1-carbonyl-CoA dehydrogenase enzyme. The cyclohexene ring of the 6-OCH-CoA may then be hydrolytically cleaved by a 6-oxocyclohex-1-ene-1-carbonyl-CoA hydrolase enzyme (a ring-opening hydrolase) to make 6-hydroxypimelyl-CoA.

The following stages of the pathway may include the enzymatic conversion of the hydroxypimelyl-CoA into Acetyl-CoA. This stage of the pathway may include a more direct decomposition of the hydroxypimelyl-CoA into carbon dioxide and Acetyl-CoA. Alternatively, the hydroxypimelyl-CoA may be converted to Acetyl-CoA through a series of intermediates in a β-oxidation like pathway. For example, 3-hydroxypimelyl-CoA may be converted to 3-ketopimelyl-CoA through an enzymatic conversion using a short chain alcohol dehydrogenase. The 3-ketopimelyl-CoA may then be enzymatically decomposed into Glutaryl-CoA and a first Acetyl-CoA using an acyl-CoA acetyltransferase enzyme. The Glutaryl-CoA may then be enzymatically converted to Crotonyl-CoA using glutaryl-CoA dehydrogenase and glutaconyl-CoA decarboxylase. The Crotonyl-CoA may be enzymatically converted to 3-hydroxybutyryl-CoA using a 3-hydroxybutyryl-CoA dehydratase enzyme. The 3-hydroxybutyryl-CoA may then be enzymatically converted to acetoacetyl-CoA with an 3-hydroxybutyryl-CoA dehydrogenase enzyme. Finally, the acetoacetyl-CoA may be enzymatically decomposed into two more Acetyl-CoAs using a acetoacetyl-CoA thioase enzyme.

In the final stages of the enzymatic pathway, Acetyl-CoA may be converted into acetate, which may then be enzymatically decomposed into methane and carbon dioxide. These stages may include the enzymatic conversion of Acetyl-CoA into acetyl-phosphate using a phosphotransacetylase enzyme, followed by the conversion of the acetyl-phophate into acetate using an acetate kinase enzyme. The acetate generated may be finally converted into methane and carbon dioxide using a familiar metabolic pathway for anaerobic methanogens. For example, acetate may be converted to N⁵-methyl-tetrahydromethanopterin and CO₂. The N⁵-methyl-tetrahydromethanopterin may then be converted to methyl-coenzyme M (CH₃—S-CoM), which is a central intermediate in the final stages of methanogenesis. Methyl-conenzyme M is then enzymatically reacted with a second thiol coenzyme (CoB-SH) to form methane and an CoM-S—S-CoB, using the enzyme methyl-coenzyme M reductase.

As the description of FIG. 4 above indicates, a complete enzymatic pathway for the conversion of a starting aromatic hydrocarbon to a hydrogen-carbon-containing fluid like methane can take several stages, each having several steps. Additional steps (not shown) are necessary when the staring aromatic groups include polycyclic compounds whose complex ring structures may be at least partially decomposed, or substituted before the aromatic hydrocarbon is activated by the addition of a chemical group. For example, a methyl group may be added to naphthalene to form 2-methyl-naphthalene prior to a fumarate activation step. It should be appreciated that not all steps in each stage have to be followed in the order described, that one or more steps may be omitted, and that different pathways are possible for the same compound. It should also be appreciated that one or more stages may be bypassed by an alternate enzymatic pathway that can supply a compound to the remaining downstream stages.

### Exemplary Enzymatic Pathway for Non-Cyclic Hydrocarbons

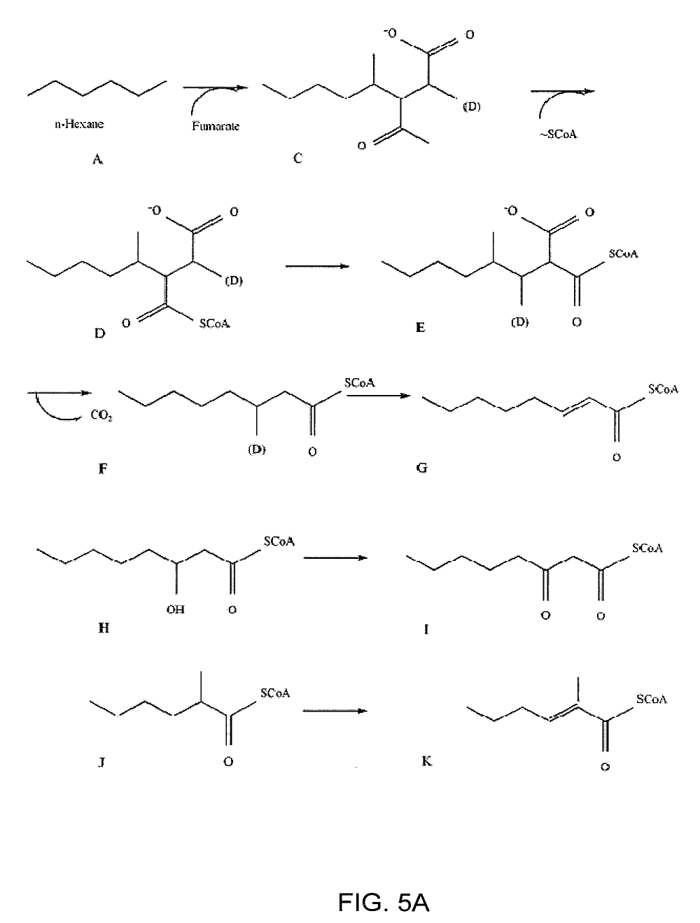

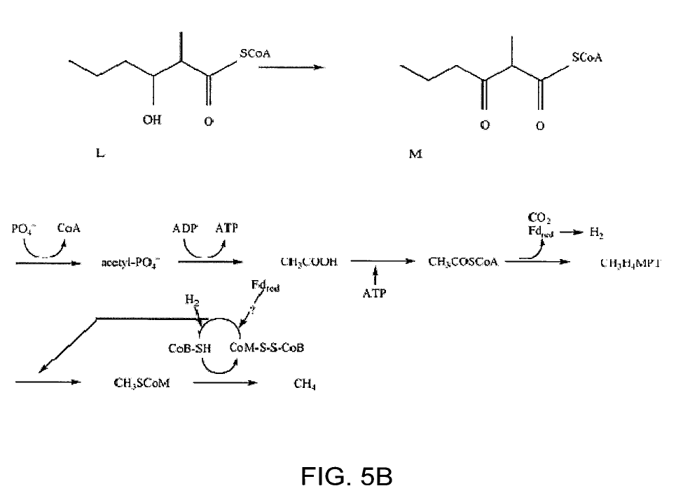

FIGS. 5A & B show an exemplary enzymatic pathway for the anaerobic metabolism of a starting non-cyclic hydrocarbon to methane (i.e., the final hydrogen-carbon-containing fluid). Non-cyclic hydrocarbons may include substituted or unsubstituted, linear or branched, saturated or unsaturated, alkanes, alkenes, and/or alkynes, among other hydrocarbons. These compounds are often components of oils, coals, shales, and tars present in geologic formations.

The first stages of this enzymatic pathway may include the addition of a chemical group to the non-cyclic hydrocarbon. For example, fumarate (HO₂CCH═CHCO₂H) (Molecule B in FIG. 5A) may be added to an alkane (e.g., n-hexane—Molecule A in FIG. 5A) to make an activated hydrocarbon. The addition of the fumarate to the hexane to form 2-(1-methylpentyl)succinate (Molecule C) may be catalyzed by a 1-methylalkyl succinate synthase enzyme.

The next stages of in the pathway may include the enzymatic conversion of the activated hydrocarbon to a fatty acid. These may include the addition of a CoA substitutent to the methylpentyl succinate to form (1-methylpentyl)succinyl-CoA (Molecule D), which may be converted to (2-methylhexyl)malonyl-CoA (Molecule E), then 4-methyloctanoyl-CoA (Molecule F), then 4-methyloct-2-enoyl-CoA (Molecule G), then 3-hydroxy-4-methyloctanoyl-CoA (Molecule H), then 4-methyl-3-oxooctanoyl-CoA (Molecule I), then 2-methylhexanoyl-CoA (Molecule J), then 2-methyl-hex-2-enoyl-CoA (Molecule K), then 3-hydroxy-2-methylhexanoyl-CoA (Molecule L), then 2-methyl-3-oxohexanoyl-CoA (Molecule M), then propionyl-CoA, and then butyryl-CoA. The butyryl-CoA may be enzymatically converted to Acetyl-CoA.

The final stages of the pathway may involve the enzymatic conversion of Acetyl-CoA into acetate, which may then be enzymatically decomposed into methane and carbon dioxide. These stages may include the enzymatic conversion of Acetyl-CoA into acetyl-phosphate using a phosphotransacetylase enzyme, followed by the conversion of the acetyl-phophate into acetate using an acetate kinase enzyme. The acetate generated may be finally converted into methane and carbon dioxide using a familiar metabolic pathway for anaerobic methanogens. For example, acetate may be converted to N⁵-methyl-tetrahydromethanopterin and CO₂. The N⁵-methyl-tetrahydromethanopterin may then be converted to methyl-coenzyme M (CH₃—S-CoM), which is a central intermediate in the final stages of methanogenesis. Methyl-conenzyme M is then enzymatically reacted with a second thiol coenzyme (CoB-SH) to form methane and an CoM-S—S-CoB, using the enzyme methyl-coenzyme M reductase.

Having described several embodiments, it will be recognized by those of skill in the art that various modifications, alternative constructions, and equivalents may be used without departing from the spirit of the invention. Additionally, a number of well-known enzymatic pathways and biochemical processes and elements have not been described in order to avoid unnecessarily obscuring the present invention. Accordingly, the above description should not be taken as limiting the scope of the invention.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included.

As used herein and in the appended claims, the singular forms “a”, “an”, and “the” include plural referents unless the context clearly dictates otherwise. Thus, for example, reference to “a process” includes a plurality of such processes and reference to “the enzyme” includes reference to one or more enzymes and equivalents thereof known to those skilled in the art, and so forth.

Also, the words “comprise,” “comprising,” “include,” “including,” and “includes” when used in this specification and in the following claims are intended to specify the presence of stated features, integers, components, or steps, but they do not preclude the presence or addition of one or more other features, integers, components, steps, acts, or groups.

What is claimed is:

1. A process for biogenic production of a hydrogen-carbon-containing fluid from a hydrocarbon containing formation, the process comprising providing in the formation an anaerobic microorganism consortium containing one or more enzymes to activate a starting aromatic hydrocarbon by an addition of a chemical group to the starting aromatic hydrocarbon; and
converting the activated aromatic hydrocarbon into the hydrogen-carbon-containing fluid through one or more intermediate hydrocarbons; and
recovering the hydrogen-carbon-containing fluid from the formation.

2. The process of claim 1, wherein the starting aromatic hydrocarbon is activated by a fumarate addition, and the chemical group is fumarate.

3. The process of claim 1, wherein the starting aromatic hydrocarbon is activated by a hydroxylation reaction, and the chemical group is a hydroxyl group.

4. The process of claim 1, wherein the starting aromatic hydrocarbon is activated by a methylation reaction, and the chemical group is a methyl or carboxyl group.

5. The process of claim 1, wherein the microorganism consortium contains one or more additional enzymes to convert the activated aromatic hydrocarbon into benzoyl-CoA.

6. The process of claim 1, wherein the microorganism consortium contains one or more additional enzymes to convert benzoyl-CoA into pimelyl-CoA.

7. The process of claim 1, wherein the microorganism consortium contains one or more additional enzymes to convert pimelyl-CoA into 3-hydroxypimelyl-CoA.

8. The process of claim 1, wherein the microorganism consortium contains:
a dearomatizing benzoyl-CoA reductase enzyme to convert the benzoyl-CoA into cyclohexa-1,5-diene-1-carbonyl-CoA; and
a hydratase enzyme to convert the cyclohexa-1,5-diene-1-carbonyl-CoA into 6-oxocyclohex-1-ene-1-carbonyl-CoA; and
a hydrogenase enzyme to convert the 6-oxocyclohex-1-ene-1-carbonyl-CoA into 3-hydroxypimelyl-CoA.

9. The process of claim 1, wherein the microorganism consortium contains one or more additional enzymes to convert 3-hydroxypimelyl-CoA into acetyl-CoA and CO₂.

10. The process of claim 9, wherein the microorganism consortium contains a short chain alcohol dehydrogenase enzyme to convert the 3-hydroxypimelyl-CoA into 3-Ketopimelyl-CoA.

11. The process of claim 10, wherein the microorganism consortium contains an acyl-CoA acetyltransferase enzyme to convert the 3-Ketopimelyl-CoA into Glutaryl-CoA.

12. The process of claim 11, wherein the microorganism consortium contains glutaryl-CoA dehydrogenase and glutaconyl-CoA decarboxylase enzymes to convert the Glutaryl-CoA into Crotonyl-CoA.

13. The process of claim 12, wherein the microorganism consortium contains a 3-hydroxybutyryl-CoA dehydratase enzyme to convert the Crotonyl-CoA into 3-hydroxybutyryl-CoA.

14. The process of claim 13, wherein the microorganism consortium contains a 3-hydroxybutyryl-CoA dehydrogenase enzyme to convert the 3-hydroxybutyryl-CoA into acetoacetyl-CoA.

15. The process of claim 14, wherein the microorganism consortium contains a acetoacetyl-CoA thiolase to convert the acetoacetyl-CoA into acetyl-CoA.

16. The process of claim 1, wherein the microorganism consortium contains one or more additional enzymes to convert acetyl-CoA into acetate.

17. The process of claim 1, wherein the microorganism consortium contains one or more additional enzymes to convert acetate into methane and CO₂.

18. The process of claim 1, wherein the hydrogen-carbon-containing fluid comprises an alkane having 1 to 5 carbon atoms.

19. The process of claim 1, wherein the hydrogen-carbon-containing fluid comprises an alcohol or an organic acid.

20. A process for biogenic production of a hydrogen-carbon-containing fluid from a hydrocarbon containing formation, the process comprising stimulating in the formation an anaerobic microorganism consortium containing one or more enzymes to activate a starting aromatic hydrocarbon by an addition of a chemical group to the starting aromatic hydrocarbon; and
converting the activated aromatic hydrocarbon into the hydrogen-carbon-containing fluid through one or more intermediate hydrocarbons; and
recovering the hydrogen-carbon-containing fluid from the formation.

21. A process for biogenic production of a hydrogen-carbon-containing fluid from a hydrocarbon containing formation, the process comprising providing to the formation an anaerobic microorganism consortium containing one or more enzymes to activate a starting alkane into an activated hydrocarbon by an addition of a chemical group to the starting alkane; and
converting the activated hydrocarbon into the hydrogen-carbon-containing fluid through one or more intermediate hydrocarbons; and
recovering the hydrogen-carbon-containing fluid from the formation.

22. The process of claim 21, wherein the starting alkane is activated by a fumarate addition, and the chemical group is fumarate.

23. The method of claim 22, wherein the starting activated hydrocarbon is a substituted succinate.

24. The process of claim 21, wherein the starting alkane is a C₆ to C₄₀ alkane.

25. The process of claim 21, wherein the microorganism consortium contains one or more additional enzymes to convert the activated hydrocarbon into a fatty acid.

26. The process of claim 25, wherein the microorganism consortium contains one or more additional enzymes to convert the fatty acid into acetate.

27. The method of claim 26, wherein the microorganism consortium contains one or more additional enzymes to convert the acetate into methane and CO₂.

28. The method of claim 21, wherein the hydrogen-carbon-containing fluid comprises methane.

29. A process for biogenic production of a hydrogen-carbon-containing fluid from a hydrocarbon containing formation, the process comprising stimulating in the formation an anaerobic microorganism consortium containing one or more enzymes to activate a starting alkane into an activated hydrocarbon by an addition of a chemical group to the starting alkane; and
converting the activated hydrocarbon into the hydrogen-carbon-containing fluid through one or more intermediate hydrocarbons; and
recovering the hydrogen-carbon-containing fluid from the formation.

30. A process for biogenic production of a hydrogen-carbon-containing fluid from a hydrocarbon containing formation, the process comprising identifying a gene encoding a target enzyme in an anaerobic enzymatic pathway to convert a starting hydrocarbon into the hydrocarbon-carbon-containing fluid;
providing a microorganism containing the identified gene to an anaerobic microorganism consortium, wherein the consortium contains one or more enzymes to activate the starting hydrocarbon into an activated hydrocarbon by an addition of a chemical group to the starting hydrocarbon, and converting the activated hydrocarbon into the hydrogen-carbon-containing fluid through one or more intermediate hydrocarbons; and
recovering the hydrogen-carbon-containing fluid from the formation.

31. The process of claim 30, wherein the starting hydrocarbon comprises an aromatic hydrocarbon or an alkane.
